(19) United States
(12) Patent Application Publication
(10) Pub. No.: US 2011/0212198 A1
(43) Pub. Date: Sep. 1, 2011

(54) HANGOVER RELIEF BY COMPOSITIONS COMPRISING ORAL REHYDRATION SOLUTION

(75) Inventors: Louis Vermeulen, Amsterdam (NL); Joost Jacobus Cornelis Verhoeff, Harderwijk (NL)

(73) Assignee: Academishsch Ziekenhuis Bij de Universiteit van Amsterdam, Amsterdam (NL)

(21) Appl. No.: 13/062,125

(22) PCT Filed: Sep. 4, 2009
(86) PCT No.: PCT/NL09/50533

§ 371 Date: Apr. 29, 2011

(30) Foreign Application Priority Data
Sep. 4, 2008 (EP) 08163642.5

(51) Int. Cl. A61K 36/28; A61P 29/00; A61P 1/08; A61P 1/14

(52) U.S. Cl. 424/764

(57) ABSTRACT

A composition comprising an oral rehydration solution and Yacón Syrop, is described. Such composition may be used as a medicament, in particular for preventing or treating one or more symptoms of a hangover, such as a headache, nausea and stomach ache.

### BACKGROUND

A hangover describes the unpleasant physiological effects following heavy alcohol consumption. The most commonly reported symptoms of a hangover include headache, nausea, and a diminished feeling of general well-being. The symptoms vary from person to person, and occasion to occasion, usually beginning several hours after drinking. It is believed that the economic impact of hangovers is severe, and estimates run as high as 2-5% of the gross-national product, mostly due to decreased productivity the day after extensive alcohol intake (Wiese et al. (2000). Ann. Intern. Med. 132, 897).

Pathophysiology of hangover is largely unknown. Hypoglycemia, dehydration, acetaldehyde intoxication and vitamin B12 deficiency are all theorised causes of hangover symptoms. It is clear from studies that during the hangover period at least dehydration, cytokine dysregulation and gastrointestinal disturbances occur, and levels of anti-diuretic hormone and prostaglandins are elevated (Verster (2008). Alcohol Alcohol 43, 124; Pittler et al. (2005). B M J 331, 1515). In addition to alcohol, impurities present in alcoholic beverages seem to contribute to the severity of alcohol symptoms. A wide spectrum of potential cures and preventives have been studied, with very limited, if any effect (Wiese et al., supra, and Pittler et al., supra).

### SUMMARY OF THE INVENTION

The present inventors have now found that fast replenishment of fluids and electrolytes by ingestion of a composition comprising a modified oral rehydration solution after more than moderate alcohol consumption reduces hangover symptoms.

Thus, the invention relates to a composition comprising an oral rehydration solution.

In a further aspect, the invention relates to a composition comprising an oral rehydration solution for use as a medicament, in particular for preventing or treating one or more symptoms of a hangover.

### DESCRIPTION OF THE DRAWINGS

The invention will be further elucidated referring to the following figures:

FIG. 1a shows the distribution of hangover severity as measured by a-AHS for the control (grey line), for placebo-treated group (dashed line) and for ORS-treated group (black line). Data are represented as histograms normalised for bin displaying highest number (% of maximum) in every group. The lines display Gaussian curves based on the data. FIG. 1b shows that overlay of Gaussian curves illustrates apparent reduced hangover symptoms after ORS intake. Categories (none, mild, severe and very severe hangover) were deducted from comparison of a-AHS score and experienced hangover severity of placebo-treated participants.

FIG. 2 shows that ORS relieves hangover symptoms at different levels of alcohol intake. In both categories, 1.0-2.4% and 2.5-4.0% estimated blood alcohol concentrations (BAC), respectively, hangover symptoms, as indicated by a-AHS, are reduced at a similar rate.

FIG. 3 shows subjective score for items loss of appetite and nausea of the a-AHS after consumption of ORS or ORS+Yacon-syrup (ORS+Y).

### DETAILED DESCRIPTION OF EMBODIMENTS

The present inventors have found that an oral rehydration solution may be used to prevent or treat one or more symptoms of a hangover. Thus, the present invention relates to an oral rehydration solution or a composition comprising an oral rehydration mixture, in particular for preventing or treating one or more symptoms of a hangover.

Symptoms of a hangover include, without limitation, thirst, headache, nausea, tiredness, loss of appetite, stomach ache, dizziness, faintness, and the like.

An oral rehydration solution (“ORS”) is an aqueous liquid comprising a glucose containing saccharide and sodium ions. The ORS has a total osmolarity of from 200 to 350 mosmol/L. Preferably, the ORS comprises from 35 to 90 milliequivalents per Liter (meq/L) of sodium (Na⁺) and from 1.2 to 3.0 weight percent (wt %) of a glucose containing saccharide.

The glucose containing saccharide may be glucose, or a saccharide that can be hydrolyzed to form a composition containing glucose. Further non-limiting examples of such glucose containing saccharides are dextrose, fructooligosaccharides, fructose, corn syrup, high fructose corn syrup, sucrose, maltodextrin, and mixtures thereof.

The ORS may further include potassium. The concentration of potassium ions may, for example, be in the range of 0 to 40 mmol/L. The ORS may further comprise chloride ions. The concentration of chloride ions may be in the range of 0 to 100 mmol/L. The ORS may further comprise citrate and/or bicarbonate.

The ORS may also comprise one ore more artificial sweeteners. Non-limiting examples of such artificial sweeteners are sodium saccharine, aspartame, acesulfame-K and chlorinated sucrose.

The ORS may also comprise one or more additional ingredients. Non-limiting examples of additional ingredients include flavourants, colourants, preservatives, dietary supplements, and the like.

The oral rehydration solution is advantageously prepared by combining the non-aqueous (“dry”) ingredients with an aqueous liquid, e.g. water, to provide a solution having the appropriate concentrations of ingredients. The dry ingredients may be combined with the aqueous liquid simultaneously, or one or more of the dry ingredients may be added separately. It may be desirable to provide all or most of the dry ORS ingredients in a single composition, referred to as an oral rehydration mixture (“ORM”). In particular, an ORM may comprise the glucose containing saccharide and sodium ions, and optionally also further ingredients used to prepare the oral rehydration solution according to the present invention.

The composition of the present invention may further comprise one or more booster additive selected from the group consisting of Yacón Syrop and vitamins.

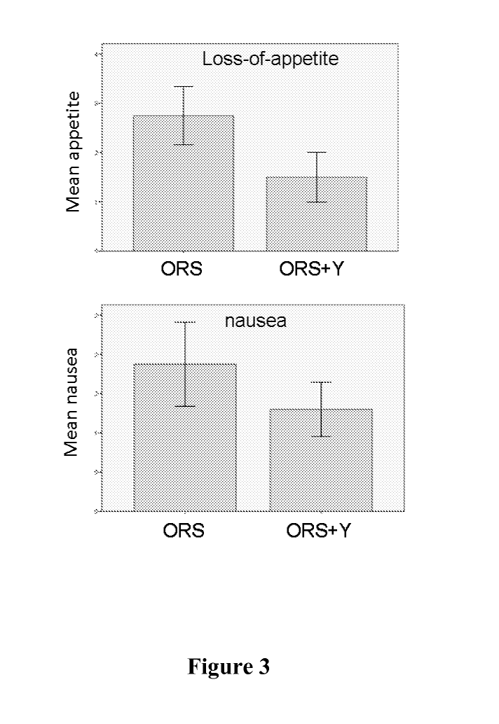

In an embodiment, the booster additive is Yacón syrup. Yacón is a sweetening agent extracted from the tuberous roots of the yacón plant (Smallanthus sonchifolius), indigenous to the Andes mountains. It contains high levels of fructooligosaccherides (FOS) (Valentova, K., Sersen, F., and Ulrichova, J. (2005). Radical scavenging and anti-lipoperoxidative activities of Smallanthus sonchifolius leaf extracts. J. Agric. Food Chem. 53, 5577-5582). It is reported that FOS derived from Yacon can lower blood sugar levels and therefore, the present inventors speculated, relief nausea related symptoms (Aybar, M. J., Sanchez Riera, A. N., Grau, A., and Sanchez, S. S. (2001). Hypoglycemic effect of the water extract of Smallantus sonchifolius (yacon) leaves in normal and diabetic rats. J. Ethnopharmacol. 74, 125-132; Genta, S., Cabrera, W., Habib, N., Pons, J., Carillo, I. M., Grau, A., and Sanchez, S. (2009). Yacon syrup: beneficial effects on obesity and insulin resistance in humans. Clin. Nutr. 28, 182-187). In addition, extracts from the Yacon root contain high amounts of anti-oxidants that might be beneficial in prevention and/or recovery from the hangover syndrome and might reduce late-time effects of more then moderate alcohol consumption (Yan, X., Suzuki, M., Ohnishi-Kameyama, M., Sada, Y., Nakanishi, T., and Nagata, T. (1999). Extraction and identification of antioxidants in the roots of yacon (Smallanthus sonchifolius). J. Agric. Food Chem. 47, 4711-4713; Valentova et al., supra). It was observed by the present inventors that loss of appetite was significantly less in subjects receiving ORS comprising Yacon syrup compared to subject receiving ORS not comprising Yacon syrup. A similar, but small, trend was observed for the item nausea (see FIG. 3). ORS comprising Yacon syrup seems to be a better and more robust anti hangover product than is ORS. Finally, we would like to emphasize that addition of Yacon-syrup to ORS enhances the palatability of the product.

The composition according to the present invention may further comprise vitamin C. Vitamin C may be added as an antioxidant to scavenge free radicals in the body, e.g. resulting from alcohol ingestion.

It has been hypothesized that alcohol may cause vitamin deficiencies, in particular of B vitamins. B vitamins are needed to help the body cope with stress, including physical stress from exposure to ethanol, its metabolites, methanol (found in red wine and cognac), and sulphites (used to preserve many beverages). Riboflavin (vitamin B2) helps the body to use oxygen and regulates the metabolism of amino acids, fatty acids, and carbohydrates. Riboflavin is further needed to activate pyridoxine (vitamin B6). Pyridoxine (vitamin B6) balances the hormonal changes in women as well as assisting the immune system and the growth of new cells. Pyridoxine also assists in the processing and metabolism of proteins, fats and carbohydrates and with controlling mood and behaviour. Cyanocobalamine (vitamin B12) guides the manufacture and maintenance of red blood cells, stimulates appetite, promotes growth and releases energy.

Thus, in an embodiment the composition according to the present invention further comprises vitamin B2, vitamin B6 and/or vitamin B12 to prevent the occurrence of vitamin deficiencies after heavy alcohol consumption.

In a further aspect, the present invention relates to a composition as described above for use as a medicament, in particular for preventing or treating one or more symptoms of a hangover. The one or more symptoms preferably include at least headache, nausea and stomach ache.

The composition according to the present invention may be further used to prevent or treat any condition involving dehydration, e.g. diarrhea, shock, and the like.

It will be clear to the skilled person that the above description and drawings are included to illustrate the invention, and not to limit the scope of protection in any way.

### EXAMPLES

### Example 1

### Materials and Methods

Prior to participation all subjects were asked for informed consent. Solutions were prepared with mineral water (Bar-Le-Duc, Utrecht, The Netherlands).

The ORS used (Orisel®, Nutricia, Zoetermeer, The Netherlands) has the following composition: glucose 20.0 g/L, Na⁺: 2.06 g/L, K⁺: 0.79 g/L, Cl⁻: 2.83 g/L; osmolarity: 320 mOsmol/L. The taste of both ORS and placebo-solution was masked with aspartame (4 g/L in ORS or 6 g/L in placebo solution). Solutions were bottled in HDPE 500 mL bottles (FLEPL1072, Bloklandpack, IJsselstein, The Netherlands).

The Acute Hangover Scale as described by Rohsenow et al. (Rohsenow et al. (2007). Addict. Behav. 32, 1314) was adapted for the purpose. The adapted-AHS (a-AHS) consists of the average of eight items that can be scored from 1 (absent) up to 7 (incapacitating): 1. hangover 2. thirsty 3. tiredness 4. headache 5. loss of appetite 6. stomach ache 7. nausea 8. dizziness, faintness. From the original AHS the inventors omitted the item ‘heart racing’ because it was very rare in our study population and changed the scaling in order to get a more robust indication of hangover severity. The effect sizes of Control group versus Placebo group match with first description of AHS, confirming the validity of our a-AHS. We asked the participants of the study to fill out a questionnaire shortly after waking up the morning after the trial. The control group was asked to fill out a similar score sheet on a ‘regular’ morning.

### Blood/Breath Alcohol Concentration:

We obtained an indication of blood alcohol levels by assessing the volunteers' breath alcohol percentage (AlcoScan Pro, AL7000, ACE Germany).

### Statistics:

Statistics were performed with SPSS 15.0 for windows and GraphPad Prism 4.0 as described in Table S1. For effect size evaluation we calculated Cohen d values;

$$d = \frac{{mean}_{1} - {mean}_{2}}{\sqrt{{SD}_{1}^{2} + {SD}_{2}^{2} / 2}} .$$

Cohen roughly defined effect sizes as “small, d=0.2” “medium, d=0.5” and “large, d=0.8”. (J. Cohen, Statistical power analysis for the behavioural sciences, 1988, 2^{nd} ed.)

### Results

Trial participants were recruited at social events where relatively high levels of alcohol are consumed. At arrival, when sober, potential participants were informed on trial procedures and gave informed consent. On leaving the event, subjects were randomly given either 500 ml ORS sweetened with aspartame or an equivalent amount of aspartame sweetened water. The present inventors used aspartame to minimize taste differences in order to ascertain the blind nature of the study. In addition, the present inventors established a blood alcohol concentration of a subset (n=39) of our participants at the moment they left the party (range 1.10-3.60%). For characteristics of participants see Table 1. Subjects were asked to fill out an online questionnaire the morning after, to evaluate hangover severity (see Table 1). For this purpose the Acute Hangover Scale (AHS) as previously described by Rohsenow et al. (Rohsenow et al. (2007). Addict. Behay., vol. 32:1314) was adapted. The adapted-AHS (a-AHS) averages eight items (i.e. headache, thirst, nausea) that can be scored from 1 (absent) up to 7 (incapacitating).

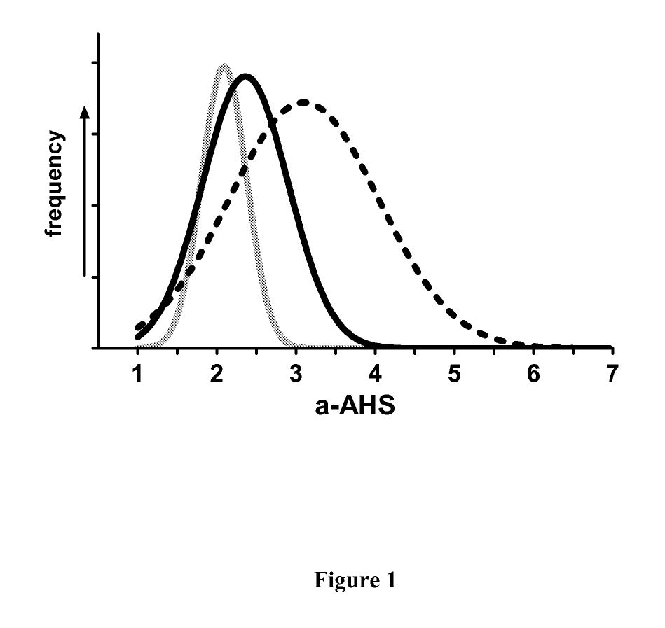

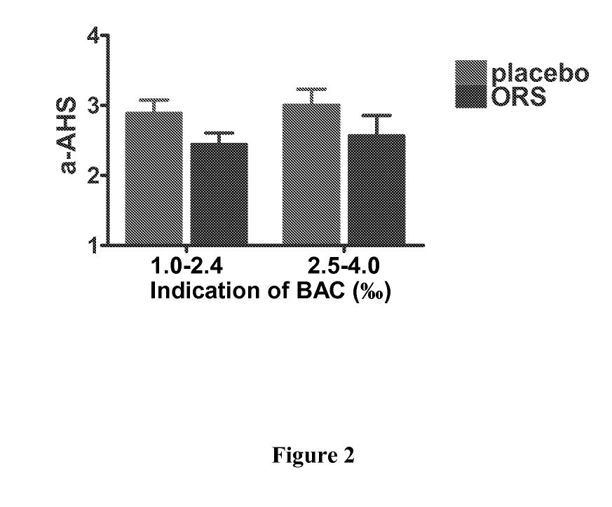

The inventors established that intake of ORS after alcohol consumption significantly reduced the hangover severity in our studied population (mean a-AHS score 3.04 in placebo-treated population and 2.39 for ORS-treated group, p<0.001) (FIG. 1). This was mainly due to reduced scores on the items ‘headache’ (p=0.007) and ‘stomach ache’ (p=0.013). To investigate if the marked relief we found is of importance for general hangover experience the inventors asked the subjects how they would rate their hangover compared to previous ones experienced. Here the inventors also found a significant reduction (see Table 1; p<0.001). In addition, we calibrated the a-AHS by inclusion of a control population not subjected to extensive alcohol consumption the night before. Strikingly, intake of ORS reduces the a-AHS almost to calibrated baseline levels (FIG. 1). An unexpected finding was a significantly higher response rate in the ORS-treated group as compared to the placebo group (78% vs. 60%; p=0.035). This difference was only observed in the male population (79% vs. 48%; p=0.004), and could indicate an increase in general performance levels. It was also found that ORS relieves hangover symptoms at different levels of alcohol intake (FIG. 2). In both categories, 1.0-2.4% and 2.5-4.0% estimated blood alcohol concentrations (BAC), respectively, hangover symptoms, as indicated by a-AHS, were reduced at a similar level.

In conclusion, drinking 500 ml of ORS prior to sleep after extensive alcohol consumption is beneficial for prevention of hangover associated symptoms. This supports the idea that dehydration is a pivotal pathophysiological mechanism in hangover occurrence, which has until now, never been scientifically proven (Verster. (2008). Alcohol Alcohol, vol. 43:124). In addition, this might provide an easy, cheap and safe way of reducing the discomfort of the hangover, and associated public expenses. It did not escape our notice, however, that knowledge of the preventive effect of ORS might increase alcohol intake, and therefore further research to this end is warranted.

TABLE 1
Participant Characteristics and Results

| Characteristics | Control | Placebo | ORS | Significance |
|---|---|---|---|---|
| Volunteers (n) | 52 | 67 | 59 | N.A. |
| Age (yr.) | 21.3 ± 2.1 | 23.7 ± 6.1 | 22.0 ± 2.6 | p = 0.401^{†} |
| (mean ± SD) | | | | |
| Sex (% male) | 52.3 | 59.7 | 64.4 | p = 0.28^{‡} |
| BAC* (mean ‰) | N.A. | 2.30 | 2.13 | p = 0.387^{†} |
| (range) | | (1.10-3.60) | (1.60-3.20) | |

| | | | | Effect size | Effect size |
|---|---|---|---|---|---|
| | | | | (control vs. | (placebo vs. |
| Results^{#} | Control | Placebo | ORS | placebo)^{Ω} | ORS)^{Ω} |
| Thirsty | 3.60 ± 1.47 | 3.75 ± 1.19 | 3.04 ± 1.40 | 0.11; p = 0.036 | 0.55; p = 0.013 |
| Headache | 1.63 ± 1.03 | 3.30 ± 1.74 | 2.35 ± 1.43 | 1.17; p < 0.001 | 0.60; p = 0.007 |
| Nausea | 1.31 ± 0.64 | 2.08 ± 1.33 | 1.67 ± 0.97 | 0.74; p < 0.001 | 0.35; p = 0.110 |
| Tiredness | 3.81 ± 1.46 | 4.15 ± 1.46 | 3.74 ± 1.42 | 0.23; p = 0.850 | 0.28; p = 0.191 |
| Loss of appetite | 1.81 ± 1.46 | 2.63 ± 1.58 | 2.24 ± 1.46 | 0.54; p = 0.149 | 0.26; p = 0.243 |
| Hangover | 1.00 ± N.A. | 3.55 ± 1.32 | 2.59 ± 1.11 | 2.73; p < 0.001 | 0.79; p < 0.001 |
| Stomach ache | 1.23 ± 0.47 | 2.43 ± 1.55 | 1.70 ± 1.11 | 1.05; p < 0.001 | 0.54; p = 0.013 |
| Dizziness, faintness | 1.40 ± 0.57 | 2.48 ± 1.49 | 1.87 ± 1.11 | 0.96; p < 0.001 | 0.46; p = 0.034 |
| a-AHS score | 1.97 ± 0.43 | 3.04 ± 0.90 | 2.39 ± 0.59 | 1.52; p < 0.001 | 0.85; p < 0.001 |
| Hangover severity | N.A. | 3.58 ± 1.06 | 2.63 ± 1.12 | N.A. | 0.87; p < 0.001 |
| compared to | | | | | |
| previous ones | | | | | |

*Indication of Blood Alcohol Concentration as derived from breath alcohol concentration prior to treatment in a subset
^{†}Student's T-test for significance between Placebo and ORS-treated groups (p-value)
^{$}Fraction of volunteers that completed the on-line response form the morning after
^{‡}χ² -test for significance between Placebo and ORS groups (p-value)
^{Ω}Effect size calculation (Cohen d value; p-value Student's T-test) d = 0.50-0.79 medium effect, d > 0.8 large effect (see supplemental material and methods for details.)
^{#}Items of the adapted-Acute Hangover Scale (a-AHS) (mean ± SD). Data based on volunteers that completed the online response form

### Example 2

A formulation of ORS further comprising Yacon-syrup was tested in a population of 18 healthy young volunteers that were administered 500 ml of either ORS or ORS+1% Yacon-syrup (ORS^{+Y}) after an evening of high alcohol intake at a social event. The next morning the volunteers filled out the online a-AHS form and both groups were compared.

A significant reduction in the score on the item loss of appetite was observed in the ORS^{+Y} treated group compared to the control population (FIG. 3). A similar, but small, trend was observed for the item nausea. Based on these results it is concluded that ORS^{+Y} is a better and more robust anti hangover product than is ORS. Finally, addition of Yacon-syrup to conventional ORS enhanced the palatability of the product considerably.

1. Composition comprising an oral rehydration solution and Yacón Syrop.

2. Composition according to claim 1, further comprising vitamin C.

3. Composition according to claim 1, further comprising vitamin B2.

4. Composition according to claim 1, further comprising vitamin B6.

5. Composition according to claim 1, further comprising vitamin B12.

6. Composition according to claim 1, for use as a medicament.

7. Composition according to claim 1, for use as a medicament for preventing or treating one or more symptoms of a hangover.

8. Method of using an oral rehydration solution, preferably a composition according to claim 1, in the preparation of a medicament for preventing or treating one or more symptoms of a hangover.

9. Composition according to claim 2, further comprising vitamin B2.

10. Method of using an oral rehydration solution, preferably a composition according to claim 1, for preventing or treating one or more symptoms of a hangover.
